(19) United States
(12) Patent Application Publication
(10) Pub. No.: US 2025/0025495 A1
(43) Pub. Date: Jan. 23, 2025

(54) Diagnosis and Treatment of Metabolic Syndrome with Magnesium Compositions

(71) Applicants: UNM Rainforest Innovations, Albuquerque, NM (US); THE UNITED STATES GOVERNMENT AS REPRESENTED BY THE DEPARTMENT OF VETERANS AFFAIRS, Washington, DC (US)

(72) Inventors: Henry C. Lin, Albuquerque, NM (US); Nancy L. Kanagy, Albuquerque, NM (US); Aleksandr Birg, Albuquerque, NM (US); Sudha Singh, Albuquerque, NM (US); Cristina N. Coffman, Albuquerque, NM (US)

(21) Appl. No.: 18/775,053

(22) Filed: Jul. 17, 2024

Related U.S. Application Data
(60) Provisional application No. 63/527,374, filed on Jul. 18, 2023.

(51) Int. Cl. A61K 33/06; A61K 31/198

(52) U.S. Cl. CPC A61K 33/06; A61K 31/198

(57) ABSTRACT

The present invention is directed to compositions which comprise at least two components comprising a magnesium compound and glutamine, often three components, a high bioavailability magnesium salt or chelate, a low bioavailability magnesium salt or chelate and glutamine (often as a neutral species or as a zwitterionic salt). It has been discovered unexpectedly that the compositions which contain these two or three components are effective at treating/inhibiting metabolic syndrome and its symptomology, including insulin resistance, and related disease states and/or conditions. Compositions according to the present invention also inhibit/treat dysbiosis such that the gut microbiome is maintained or returns to normal.

### FIELD OF THE INVENTION

The present invention is directed to compositions which comprise two components comprising a magnesium compound and glutamine, often three components, a high bioavailability magnesium salt or chelate, a low bioavailability magnesium salt or chelate and glutamine (often as a neutral species or as a zwitterionic salt). It has been discovered unexpectedly that the compositions which contain these three components are effective at treating/inhibiting metabolic syndrome and its symptomology (e.g. impaired glucose metabolism, insulin resistance, dysregulation of blood pressure and dyslipidemia, thus having a favorable effect on cardiovascular disease, diabetes (especially diabetes II), cerebrovascular disease, fatty liver disease, especially non-alcoholic fatty liver disease and non-alcoholic steatohepatitis (NASH) and fibrosis, including cirrhosis. Compositions according to the present invention also inhibit/treat dysbiosis such that the gut microbiome is maintained or returns to normal. The chemical composition and methods of the present invention are thus useful in treating both metabolic syndrome and its related disease states, conditions and symptomology as well as gut dysbiosis.

### RELATED APPLICATIONS

This application claims the benefit of priority of U.S. provisional application Ser. No. 63/527,374, filed Jul. 18, 2023, the entire contents of which application is incorporated by reference herein.

### BACKGROUND AND OVERVIEW OF THE INVENTION

Metabolic Syndrome and Dysbiosis: Metabolic syndrome (MetS), a condition of low grade systemic inflammation, is a universally accepted disease process that describes impaired glucose metabolism, insulin resistance, dysregulation of blood pressure and dyslipidemia. The exact definition and cut-off values of this condition vary slightly but the umbrella definition is the similar across major guidelines as well as the World Health Organization. At least a quarter of the world population meets the diagnosis of MetS with a total cost of over a trillion dollars per year. The high prevalence of MetS is greatly concerning as it increases the risk of cardiovascular disease, diabetes, cerebrovascular disease, fatty liver disease (NAFLD) and non-alcoholic steatohepatitis (NASH). Abnormal changes to the gut microbiome, dysbiosis, are frequently reported in MetS as a contributing factor to development of complications such as insulin resistance via modulation of energy metabolism and systemic inflammatory responses but whether MetS is caused by dysbiosis is not known. Effective treatment of MetS depends on the answer.

A potential synergistic possibility is to combine magnesium with glutamine. The inventors completed a study inducing gut dysbiosis with lectin-containing raw red kidney beans (RRKB) showing that gut dysbiosis leads to leaky gut along with the adverse effect of microbial translocation to the liver. Most importantly, the inventors showed that glutamine is able to reverse these effects by inducing heat shock factor 70.

Gut Microbes, Small Intestinal Bacterial Overgrowth, Obesity, Diabetes, Fatty Liver, Metabolic Syndrome and Other Conditions Associated with Chronic Inflammation

The role of gut microbes in obesity has been supported by twin studies which are either discordant or concordant for obesity showing that fecal microbial transplant from obese but not lean human subjects resulted in obesity in recipient germ-free mice (Ridaura V K, et al. Science 2013; 341: 1241214). Reduced gut microbial diversity as a feature of gut dysbiosis was associated with progression of type 2 diabetes (Nature 2013; 500:585-588).

Correspondingly, fecal microbial transplant from healthy subjects improved insulin sensitivity in patients with type 2 diabetes (Vrieze A, et al. Gastroenterology 2012; 143: 913-916). Insulin resistance was associated with increased proteobacteria (Kootte R S, et al. Cell Metab 2017; 26: 611-619). Reduced gut microbial diversity, a characteristic feature of a disturbed gut microbiome (gut dysbiosis), was seen in patients with obesity (Breton J, et al. Microorganisms 2022; 10(2): 452), nonalcoholic steatohepatitis (NASH) and nonalcoholic fatty liver disease (NAFLD) but the responsible bacteria is unknown (Zhu L, et al. Hepatology 2013; 57: 601-609; Mouzaki M, et al. Hepatology 2013; 58: 120-127). In addition, gut dysbiosis, as characterized by small intestinal bacterial overgrowth (SIBO), is associated with NAFLD (Wijarnpreecha K, et al. Eur J Gastroenterol Hepatol 2020; 32: 601-608). SIBO Is also associated with Type 1 diabetes and Type 2 diabetes (El Kurdi B, et al. Clin Transl Gastroenterol 2019; 10: e00072; Wijarnpreecha K, et al. Eur J Gastroenterol Hepatol 2020; 32: 601-608; Malik A, et al. Eur J Clin Invest 2018; 48: e13021). Moreover, gut dysbiosis contributes to other features of metabolic syndrome such as hypertension (Yang T, et al. Hypertension 2015; 65(6): 1331-1340) and atherosclerosis (Brandsma E, et al. Circ Res 2019; 124(1): 94-100), While commensal gut bacteria are generally well tolerated by the immune system, the shift in the microbial community that is characteristic of gut dysbiosis is sufficient to trigger an immune response. It is, therefore, not surprising that dysbiosis and/or SIBO is associated with varying degrees of chronic inflammation such as Inflammatory bowel disease such as Crohn's disease and ulcerative colitis (Tamboli C P, et al. Gut 2004; 53(1): 1-4), celiac disease (Shah A, et al. J Gastroenterol Hepatol 2022; 37(10): 1844-1852), rheumatoid arthritis (Horta-Baas G, et al. J Immunol Res 2017; 2017: 4835189, doi: 10.1155/2017/4835189), ankylosing spondyloarthritis (Sagard J, et al. Arthritis Res Ther 2022; 24: 42, doi: 10.1186/s13075-022-02733-w). systemic lupus erythromatosus (Pan Q, et al. Front Immunol 2021; 12: 799788, doi: 10.3389/fimmun.2021.799788), irritable bowel syndrome (Distrutti E, et al. World J Gastroenterol 2016; 22(7):2219-2241), uveitis (Rodriguez-Fernandez C A, et al. Int J Mol Sci 2022; 23(13): 7020, doi: 10.3390/ijms23137020), atopic disease such as allergic asthma (Barcik W, et al. Immunity 2020; 52(2); 241-255) and atopic dermatitis (Davidson W F, et al. J Allergy Clin Immunol 2019; 143(3): 894-913), eczema (Zheng H, et al. PLoS One 2016; 11(11): e0166026, doi: 10.1371/journal.pone.0166026), necrotizing enterocolitis (Kaplina A, et al. Int J Mol Sci 2023; 24(3): 2471; doi: 10.3390/ijms24032471), primary sclerosing cholangitis (Sabino J, et al. Gut 2016; 65(10): 1681-1689), primary biliary cirrhosis (Mattner J. Int J Mol Sci 2016; 17(11): 1864, doi: 10.3390/ijms17111864)m autoimmune hepatitis (Czaja A J. World J Gastroenterol 2016; 22(42): 9257-9278), alcoholic liver disease (Hyun J Y, et al. Int J Mol Sci 2022; 23(15): 8749, doi: 10.3390/ijms23158749), cirrhosis (Trebicka J, et al. J Hepatol 2021; 75(Suppl 1): S67-S81, doi: 10.1016/j.jhep.2020.11.013), colorectal cancer (Yang Y, et al. Nat Commun 2021; 12: 6757, doi: 10.1038/s41467-021-27112-y), hepatocellular carcinoma (Luo W, et al. Front Microbiol 2022; 13: 873160, doi: 10.3389/fmicb.2022.873160), attention deficit hyperactivity disorders (ADHD)(Chca-Ros A, et al. Nutrients 2021; 13(1): 249, doi: 10.3390/nu13010249), autism spectrum disorders (Alharthi A, et al. Int J Mol Sci 2022; 23(3): 1363, doi: 10.3390/ijms23031363), multiple sclerosis (Cekanaviciute E, Et al. Proc natl Acad Sci USA 2017; 114: 10713-10718), Long COVID (Hilpert K, et al. Front Microbiol 2021; 12: 732838), stroke (Chidambaram S B, et al. Cells 2022; 11(7): 1238, doi: 10.3390/cells11071239), Parkinson disease (Nowak J M, et al. Biomedicine 2022; 10(9): 2057, doi: 10.3390/biomedicines 10092057), Lewy body dementias (Ryman S, et al. J Neurol 2023; 270(2): 746-758, bipolar disorders (Dai W, et al. Front Pharmacol 2022; 13: 893567, doi: 10.3389/fphar.2022.893567), mild cognitive disorders and Alzheimer's disease (Cho J, et al. CNS Neurosci Ther 2021; 27(5): 505-514), traumatic brain injury (Hanscom M, et al. J Clin Invest 2021; 131(12): e143777, doi: 10.1172/jci143777), post-traumatic stress disorder (PTSD)(Bajaj J S, et al. Am J Physiol Gastrointest Liver Physiol 2019; 317(5): G661-G669), depression (Sonali S, et al. Cells 2022; 11(8): 1362, doi: 10.3390/cells11081362), anxiety disorders (Park K, et al. Nutrients 2021; 13(3): 811, doi: 10.3390/nu13030811), Chronic fatigue syndrome (Safadi J M, et al Mol Psychiatry 2022; 27(1): 131-153), fibromyalgia (Erdrich S, et al. BMC Musculoskelet Disord 2020; 21: 181, doi: 10.1186/s12891-020-03201-9), migraine headaches (Tang Y, et al. Mol Neurobiol 2020; 57(1): 461-468), chronic pain (Cowe R, et al. Biomedicine 2022; 10(8): 1815, doi: 10.3390/biomedicines 10081815), schizophrenia (Munawar N, et al. Int J Mol Sci 2021; 22(14): 7671, doi: 10.3390/ijms22147671) and chronic kidney disease (Huang Y, et al. Front Pharmacol 2022; 13: 837500, doi: 10.3389/fphar. 2022.837500).

### Leaky Gut, Bacterial Translocation, Endotoxemia and Metabolic Syndrome

Obesity is associated with increased gut permeability (leaky gut)(Damms-Machado A, et al. Am J Clin Nutr 2017; 105: 127-135) and is associated with insulin resistance (Teixeira T F S, et al. Clin Nutr Edinb Scotl 2012; 31: 735-740). Increased intestinal permeability correlated with systemic inflammation (Genser L, et al. J Pathol 2018; 246: 217-230) while a high fat diet, obesity, type 2 diabetes and insulin resistance are all associated with endotoxmia (Cani P D, et al. Diabetes 2007; 56: 1761-1772; Lassenius M I, et al. Diabetes Care 2011; 34: 1809-1815; Pussinen P J, et al. Diabetes Care 2011; 34: 392-397; Cox A J, et al. Diabetes Metab 2017; 43: 163-166). Correspondingly, the circulatory concentration of 16S rRNA gene was increased in those who developed type 2 diabetes out of 3280 subjects who were followed for 9 years (Amar J, et al. Diabetologia 2011; 54: 3055-3061). Correspondingly, the diagnosis of Type 2 diabetes was associated with higher circulatory concentration of 16 S rRNA gene (Sato J, et al. Diabetes Care 2014; 37:2343-2350). Since high fat diet is associated with increased intestinal permeability (Moreira A P B, et al. Br J Nutr 2012; 108(5):801-809) as evidenced by increased circulatory lipopolysaccharide (endotoxin)(Majdalawieh A, et al. Int J Biochem Cell Biol 2009; 41: 1518-1525), it was not surprising that fluorescent labeled E. coli, when administered by gavage into the gastrointestinal tract, could be recovered from mesenteric lymph nodes (Burcelin R, et al. Diabetes Obes Metab 2013; 15 (Suppl 3): 61-70) demonstrating While it was known that the increased circulatory bacteria are predominately related to Proteobacteria, whether the adverse metabolic effects are driven by a specific species of gut bacteria was not known. This finding was also associated with greater risk for developing cardiovascular disease (Amar J, et al. PLoS ONE 2013; 8: e54461). These findings supported the idea that gut dysbiosis with the microbiome perturbed by a variety of factors including a high fat diet, obesity, antibiotic exposure, infectious gastroenteritis, stress, etc. could lead to increased intestinal permeability and in turn, microbial translocation from gut to systemic circulation and the triggering of local and systemic inflammation. Circulatory bacteria or bacterial products have been reported to make their way to adipose tissues (Biswas S K, et al. Cell Metab 2016; 24: 196-198; Creely S J, et al. Am J Physiol Endocrinol Metab 2007; 292: E740-E747; Vitseva O I, et al. Obesity 2008; 16; 932-937), the liver (Bergheim I, et al. J Hepatol 2008; 48: 983-992), the pancreas (Balzan S, et al. J Gastroenterol Hepatol 2007; 22: 464-471), intestine (Chimerel C, et al. Cell Rep 2014; 9: 1202-1208), muscles (Cani P D, et al. Diabetes 2007; 56: 1761-1772; Virkamaki A, et al. Endocrinology 1994; 134: 2072-2078) and brain and nervous system (Branton W G, et al. Brain, Behavior and Immunity 2023; 107: 110-123) to drive a local and systemic inflammatory response that may be responsible for symptoms and findings in metabolic syndrome including glucose intolerance/insulin insensitivity/diabetes, hyperlipidemia/fatty liver disease, non-alcoholic fatty liver disease/steatohepatitis/cirrhosis due to fatty liver disease and brain fog (impaired short term memory, difficulty with concentration and other impaired executive functions). Increased intestinal permeability contributes to other features of metabolic syndrome such as hypertension (Ntlahla E E, et al. Afr Health Sci 2021; 21(3): 1172-1184, doi: 10.4314/ahs.v21i3.26), acute coronary syndrome (Alhmoud T, et al. Hum Microb J 2019; 13: 10059, doi: 10.1016/j.humic. 2019.100059) and atherosclerosis (Kurilenko N, et al Cells 2021; 10(2): 348, doi: 10.3390/cells 10020348). Increased intestinal permeability, driven by gut dysbiosis, lead to microbial translocation and triggering of immune response to account for many conditions associated with chronic inflammation such as Inflammatory bowel disease such as Crohn's disease and ulcerative colitis (Lyer N, Corr S C. Nutrients 2021; 13(12): 4259, doi: 10.3390/nu13124259), celiac disease (Obrenovich M E M. Microorganisms 2018; 6(4): 107, doi: 10.3390/microorganisms6040107), rheumatoid arthritis (Tajik N, et al. Nat Commun 2020; 11: 1995, doi: 10.1038/s41467-020-15831-7), ankylosing spondyloarthritis (Harkins P, et al Rheumatol Adv Pract 2021; 5(3): rkab063, doi: 10.1093/raP/RKAB063). systemic lupus erythromatosus (Charoensappakit A, et al. Int J Mol Sci 2022; 23(15): 8223, doi: 10.3390/ijms23158223), irritable bowel syndrome (Hanning N, et al. Therp Adv Gastroenterol 2021; 14: 1756284821993586, doi: 10.1177/1756284821993586), uveitis (Janowitz C, et al. Invest Ophthalmol Vis Sci 2019; 60(1): 420-429, doi: 10.1167/iovs.18-24813), atopic disease such as allergic asthma (Farshchi M K, et al. J Evid Based Complementary Altern Med 2017; 22(3): 378-380, doi: 10.1177/2156587216682169) and atopic dermatitis (Sozener Z C, et al. Allergy 2022; 77(5): 1418-1449, doi: 10.1111/all.15240), eczema (Park D H, et al. Int J Mol Sci 2021; 22(8): 4228, doi: 10.3390/ijms22084228), necrotizing enterocolitis (Rvisankar S, et al. BMC Pediatr 2018; 18: 372, doi: 10.1186/s12887-018-1346-x), primary sclerosing cholangitis (Bozward A G, et al Front Immunol 2021; 12: 711217, doi: 10.3389/fimmu.2021.711217), primary biliary cirrhosis (Mattner J. Int J Mol Sci 2016; 17(11): 1864, doi: 10.3390/ijms17111864), autoimmune hepatitis (Zhang H, et al. Front Immunol 2021; 12: 624360, doi: 10.3389/fimmun.2021.624360), alcoholic liver disease (Plaza-Diaz J, et al. Int J Mol Sci 2020; 21(21): 8351, doi: 10.3390/jims21218351), cirrhosis (Fukui H. World J Hepatol 2015; 7(3): 425-442), colorectal cancer (Genua F, et al. Front Oncol 2021; 11: 626349, doi: 10.3389/fonc.2021.626349), hepatocellular carcinoma (Yu L X, Schwabe R F. Nat Rev Gastroenterol Hepatol 2017; 14(9): 527-539, doi: 10.1038/nrgastro.2017.72), attention deficit hyperactivity disorders (ADHD)(Lee S Y, et al. Children (Basel) 2023; 10(3): 513, doi: 10.3390/children10030513), autism spectrum disorders (Al-ayadhi L, et al. Gut Pathog 2021; 13: 54, doi: 10.1186/s13099-021-00448-y), multiple sclerosis (Camara-Lemarroy C R, et al. Brain 2018; 141(7): 1900-1916, doi: 10.1093/brain/awy131), Long COVID (Ancona G, et al. Front Immunol 2023; 14: 1080043, doi 10.3389/fimmun.2023.1080043), stroke (Ahmadi S, et al. JCI Insight 2020; 5(9): e132055, doi: 10.1172/jci.insight.132055), Parkinson disease (Menozzi E, et al. AnnMed 2021; 53(1): 611-625, doi: 10.1080/07853890.2021.1890330), Lewy body dementias (Ryman S, et al. J Neurol 2023; 270(2): 746-758, bipolar disorders (Zhang P, et al. Front neurosci 2022; 16: 830748, doi: 10.3389/fnins.2022.830748), mild cognitive disorders and Alzheimer's disease (Li H et al, Front Aging Neurosci 2021; 13: 671142, doi: 10.3389/fnagi.2021.671142; Bairamian D, et al. Mol Neurodegener 2022; 17: 19, doi: 10.1186/s13024-022-00522-2), traumatic brain injury (Chiu L S, Anderton R S. Eur J Neurosci 2023; 57(2): 400-418, doi: 10.1111/ejn.15892), post-traumatic stress disorder (PTSD)(Doney E, et al. Eur J Neurosci 2022; 55(9-10): 2851-2894, doi: 10.1111/ejn.15239), depression (Beurel E, et al. Neuron 2020; 107(2): 234-256, doi: 10.1016/neuron.2020.06.002), anxiety disorders (Lach G, et al. Neurotherapeutics 2018; 15(1): 36-59, doi: 10.1007/s13311-017-0585-0), Chronic fatigue syndrome (Stanculescu D, et al. Front Neurol 2021; 12: 789784, doi: 10.3389/fneur.2021.789784), fibromyalgia (Garofalo C, et al. Biomedicine 2023; 11(6): 1701, doi: 10.3390/biomedicines 11061701), migraine headaches (Liang L, et al. Food Sci Nutr 2023; 11(4): 1571-1704, doi: 10.1002/fsn3.3229), chronic pain (Dworsky-Fried Z, et al. Neurobiol Pain 2020; 7; 100045, doi: 10.1016/j.ynpai.2020.100045), schizophrenia (Kraeuter A K, et al. Front Psychiatry 2020; 11: 799, doi: 10.3389/fpsyt.2020.00799) and chronic kidney disease (Tang T, et al. Nat Rev Nephrol 2018; 1497): 442-456, doi: 10.1038/s41518-081-0018-2).

### Overgrowth of Sulfate Reducing Bacteria in Gut Dysbiosis, Leaky Gut and Magnesium

The inventors were able to identify overgrowth of sulfate reducing bacteria (SRB), a family of rare resident gut bacteria that generates hydrogen sulfide (H2S) by using hydrogen, a gaseous by-product of fermentation as a consistent finding in gut dysbiosis regardless of the manner by which the gut microbiome was perturbed (Singh S, Carroll-Portill A, Lin H C. “Desulfovibrio in the gut, the enemy within?” Submitted to Microorganisms 2023-unpublished). We found that SRB induced increased intestinal permeability via a SNAIL (a nuclear transcription factor) dependent mechanism (Singh S, et al. Front Cell Infect Microbiol. 2022 May 26; 12:882498). In addition, we found that SRB proliferates in number and increases its production of hydrogen sulfide in the presence of the stress mediator norepinephrine (Coffman C N, et al. Anaerobe. 2022 June; 75:102582). The data suggest that SRB may be the specific strain of resident bacteria that induces leaky gut. These effects, however, could be reversed with magnesium oxide (DDW 2023 abstract attached, unpublished manuscript attached).

### Intestinal Permeability and Glutamine

In burn patients, endotoxemia and increased intestinal permeability were reversed by intravenous glutamine (Peng X, et al. Burns J Int Soc Burn Inj 2004; 30: 135-139; Wang Z E, et al. Burns 2022; 48(7):1606-1917). Similar findings have been reported in other conditions of critical illness (De Souza D A, et al. Crit Care Med 2005; 33: 1125-1135; Wischmeyer P E. Curr Opinion in Clin Nutr Metab Care 2006; 9: 607-612). This beneficial effect of glutamine is through its promotion of the expression of tight junction proteins responsible for the intestinal barrier function via calcium/calmodulin-dependent kinase 2 (CaMKK2)-AMP-activated protein kinase (AMPK) signaling (Wang B, et al. J Nutr 2016; 146(3): 501-508) and enhanced chaperon function from heat shock protein 70 (HSP 70). More HSP 70 expression was stimulated by glutamine because glutamine drove the hexoamine pathway (Hamiel C R, et al. Am J Physiol 2009; 297: C1509-1519) to facilitate the O-glycosylation of heat shock factor-1 (HSF-1) the regulatory nuclear transcription factor that drives HSP 70 expression (Morrison A L, et al. Am J Physiol Cell Physiol 2006; 290: 1625-1632; Singleton K D, Wischmeyer P E. JPEN J Parent Enteral Nutr 2008; 32: 371-376). Glutamine enhanced O-glycosylated HSF-1 then translocates to the nucleus where this transcription factor activates the heat shock factor response element on the genes that encode HSP 70.

Based on the foregoing, the present invention is directed to a novel synergistic compound that comprises magnesium and glutamine in combination, often 3 components-high bioavailability magnesium, low bioavailability magnesium and glutamine. This is described in the present application. Methods of treating metabolic syndrome, including any one or more of the symptoms of metabolic symptom such as impaired glucose metabolism, insulin resistance, dysregulation of blood pressure and dyslipidemia, as well as cardiovascular disease, diabetes, cerebrovascular disease, fatty liver disease (NAFLD) and non-alcoholic steatohepatitis (NASH), among others represent additional embodiments of the present invention.

### Impact of Glucagon-Like Peptide-1

Glucagon-like peptide-1 (GLP-1) is a well-recognized hormone responsible for insulin release, gastric emptying, and inhibition of food intake for treatment of obesity (Drucker D. Molecular Metabolism 2022; 57: 101351). Recently, GLP-1 has been shown to play a significant role in improving intestinal epithelial integrity and reducing barrier permeability, specifically in the distal small intestine (Abdalgadir N et al. Microorganisms 2022, 10, 2061). Hydrogen sulfide at high concentrations has been shown to disrupt normal intestinal barrier function and display anti-inflammatory effects, interestingly this effect is not seen at lower concentrations (Blachier F, et al. Am J Physiol Gastrointest Liver Physiol 320: G125-G135, 2021). H2S at high concentrations inhibits GLP-1 secretion by inhibiting by Takeda-G-protein-receptor-5 (TGR5) in the small intestine. TGR5 is a membrane-bound G-protein coupled receptor that binds secondary bile acids from the gut lumen. Binding secondary bile acids activates TGR5 which increases GLP-1 secretion (Arora T, et al. Med 2, 553-570, May 14, 2021).

Elevated H2S concentrations released from SRB in the small intestine can lead to inhibition of TGR5 which will inhibit release of GPL-1 which can lead to disruption of the intestinal barrier. Magnesium blocks available gut luminal H2S which can lead to downstream activation of TGR5 and release of GLP-1. As GLP-1 has been shown to lead to resolution of non-alcoholic steatohepatitis (NASH) (Drucker D. Molecular Metabolism 2022; 57: 101351), using magnesium to neutralize available H2S, can further increase GLP-1 release which may provide benefit in NASH.

### BRIEF DESCRIPTION OF THE INVENTION

The present invention is directed to compositions and methods which are particularly useful in treating metabolic syndrome and dysbiosis and/or leaky gut syndrome in patients or subjects in need. In embodiments, compositions according to the present invention comprise an effective at least one magnesium compound in combination with glutamine or a pharmaceutically acceptable salt for the combined treatment of metabolic syndrome, related symptomology as described herein and separately, gut dysbiosis. The therapy is particularly effective for the treatment of metabolic syndrome which is often found in combination with and exacerbated by gut dysbiosis. In embodiments, the composition comprises three components, an effective amount of a high bioavailability magnesium compound, often a magnesium compound selected from the group consisting of magnesium-L-threonate, magnesium malate, magnesium lactate, magnesium aspartate, magnesium citrate, magnesium diglycinate, magnesium acetyl taurate and mixtures thereof, an effective amount of a low bioavailability magnesium compound, often a magnesium compound selected from the group consisting of magnesium oxide, magnesium hydroxide, magnesium glucoheptonate, magnesium gluconate, magnesium chloride, magnesium sulfate, magnesium carbonate and mixtures thereof and glutamine or a pharmaceutically acceptable salt thereof in weight ratios which provide synergistic activity in treating metabolic syndrome and related disease states and/or conditions and dysbiosis in patients or subjects in need. In embodiments, the composition often favorably comprises two components, a magnesium salt which is magnesium sulfate, magnesium carbonate or magnesium chloride, often magnesium sulfate and glutamine or a pharmaceutically acceptable salt, both included in effective amounts.

In embodiments, compositions according to the present invention are formulated in one or two part compositions, often a single part composition.

In embodiments, the composition comprises magnesium compound(s) and glutamine or a pharmaceutically acceptable salt thereof wherein said magnesium compound comprises an amount ranging from 5 mM to 50 mM (an amount ranging from about 0.6 to 6.0 grams) or 7.5 mM to 40 mM (about 0.9 to 4.8 grams), often 5 mM to 25 mM (about 0.6 to 3.0 grams) or 7.5 to 35 mM (about 0.9 to 4.1 grams), more often 10 to 25 mM (about 1.2 to 3.0 grams), 15 to 25 mM (about 1.8 to 3.0 grams), 20 to 25 mM (about 2.4 to 3.0 grams) or about 25 mM (about 3.0 grams) and said glutamine or a pharmaceutically acceptable salt thereof comprises an amount ranging, respectively from 4 mM to 50 mM (about 0.586 to 7.5 grams), 5 mM to 75 mM (about 0.73 to 11.25 grams), 10 to 50 mM (about 1.5 to 7.5 grams), 15 to 45 mM (about 2.2 to 6.5 grams) or 20 to 45 mM (about 2.92 to 6.5 grams).

In embodiments, the magnesium salt and glutamine often are included in compositions in a molar ratio ranging from about 0.35 to about 1.0 magnesium, about 0.4 to about 0.95 magnesium, about 0.45 to about 0.90 magnesium, about 0.5 to about 0.80 magnesium, about 0.55 to about 0.75 magnesium, about 0.6 to about 0.7 magnesium or about 0.625 magnesium to each 1.0 mole of glutamine included in said composition.

In embodiments, in methods of treatment, the magnesium salt and glutamine often are co-administered to patients or subjects in a molar ratio ranging from about 0.35 to about 1.0 magnesium, about 0.4 to about 0.95 magnesium, about 0.45 to about 0.90 magnesium, about 0.5 to about 0.80 magnesium, about 0.55 to about 0.75 magnesium, about 0.6 to about 0.7 magnesium or about 0.625 magnesium to each 1.0 mole of glutamine.

In embodiments, doses of magnesium and glutamine are often administered to a patient or subject from 1 to 6 times daily, often 1 to 4 times daily often in an oral or buccal dosage form.

In embodiments, the magnesium compound is selected from the group consisting of magnesium-L-threonate, magnesium malate, magnesium chloride, magnesium lactate, magnesium aspartate, magnesium citrate, magnesium diglycinate, magnesium acetyl taurate, magnesium oxide, magnesium hydroxide, magnesium glucoheptonate, magnesium gluconate, magnesium sulfate, magnesium carbonate or a mixture thereof. In embodiments, the magnesium compound is a single compound and is magnesium sulfate, magnesium carbonate or magnesium chloride, often magnesium sulfate. In embodiments, the magnesium compound is a mixture of an effective amount of a high bioavailability magnesium compound, often a magnesium compound selected from the group consisting of magnesium-L-threonate, magnesium malate, magnesium chloride, magnesium lactate, magnesium aspartate, magnesium citrate, magnesium diglycinate, magnesium acetyl taurate and mixtures thereof and an effective amount of a low bioavailability magnesium compound, often a magnesium compound selected from the group consisting of magnesium oxide, magnesium hydroxide, magnesium gluconate, magnesium glucoheptonate, magnesium sulfate, magnesium carbonate and mixtures thereof. In embodiments, the high bioavailability magnesium compound ranges from about 0.05 to about 90%, about 1 to 75%, about 1 to 50%, about 2.5 to 45%, about 5% to 35%, about 7.5 to 30% or about 50% by weight of a mixture of high bioavailability magnesium compound and low bioavailability magnesium compound.

In embodiments, the mixture of high bioavailability magnesium compound and low bioavailability magnesium compound is used to maintain effective levels of magnesium in the gut and in the bloodstream of the patient or subject in order to promote the dual activity associated with treatment of metabolic syndrome and its related disease states and/or conditions and dysbiosis and/or leaky gut syndrome.

In embodiments, the invention is directed to a method of treating metabolic syndrome, or a related symptom, disease state or symptom of metabolic syndrome comprising administering an effective amount of at least one magnesium salt in combination with an effective amount of glutamine or a pharmaceutically acceptable salt thereof. In embodiments, the method of treatment pursuant to the present invention comprises administering an amount of magnesium compound(s) ranging from 200 mg per day (often in at least two, three or four doses) to 3,000 mg per day or more, often 200, 400, 500, 800, 900, 1000, 1200, 1250 or 1550 mg and an amount of glutamine or a pharmaceutically acceptable salt thereof ranging from 400 mg to 6000 mg per day or more, often 400, 800, 1000, 1600, 1800, 2000, 2400, 2500 and 3,000 mg per day as otherwise described herein.

Without being limited by way of theory, it has been discovered that combining magnesium compounds with glutamine will produce a synergistic effect in treating metabolic syndrome and its related symptoms, disease states and/or conditions, including impaired glucose metabolism, insulin resistance, dysregulation of blood pressure, dyslipidemia, cardiovascular disease, diabetes (especially diabetes II), cerebrovascular disease, fatty liver disease, especially non-alcoholic fatty liver disease (NAFLD) and non-alcoholic steatohepatitis (NASH) and fibrosis, including cirrhosis, as well as gut dysbiosis and/or leaky gut syndrome. Evidence supports the effect of magnesium in protecting the intestinal barrier by suppressing sulfate-reducing bacteria (SRB) and inhibiting its metabolism (blocking production of hydrogen sulfide), especially for magnesium compounds which exhibit ow bioabsorption so that concentrations remain in the gastrointestinal tract of a patient or subject. A poorly absorbable form of magnesium (e.g. magnesium oxide, magnesium sulfate, magnesium chloride, magnesium gluconate and magnesium carbonate, among others) often work well against SRB in the gut even without systemic entry as the desired effect is localized to the intestinal lumen. In contrast, an easily absorbable form of magnesium (e.g., magnesium-L-threonate, magnesium malate, magnesium citrate, magnesium diglycinate, magnesium acetyl taurate) would be more effective in achieving a systemic effect such as blocking pain triggered by bacteria-derived hydrogen sulfide as hydrogen sulfide works as a gaseous neurotransmitter in triggering the firing of pain pathways. In the case of glutamine, this compound has been reported to protect against the development of leaky gut in critical illness where a functional state of insufficient glutamine availability is common. The combined effects of magnesium with glutamine provide a particularly effective therapy for metabolic syndrome, and its related symptoms, disease states and/or conditions which are synergistic in nature.

These and/or other embodiments of the present invention may be readily gleaned from a review of the description of the invention which is provided herein.

### BRIEF DESCRIPTION OF THE FIGURES

FIG. 1 shows the results of a first in-vitro experiment that the combination of mg25+glu40 was more protective than other combinations or either Mg or Glutamine alone. The results presented suggest that the optimal molar ratio of magnesium to glutamine is 0.625:1.0. Conclusion from data presented in FIG. 1 is that the novel combination of magnesium and glutamine is more protective against leaky gut than either magnesium or glutamine alone.

FIGS. 2A and 2B show the results of a second in-vitro experiment, in which bacteria was grown with varying doses of MgO (0.5-2.0 mg/ml) in liquid media at 37° for 24 hours (n=3). Total counts were obtained and represented as cells per ml. This is shown in FIG. 2A. To account for the heavy right skew of the response variable, the analysis was conducted using a shifted log-transformation of the data and 90% confidence intervals were constructed using Cox's method. Hydrogen sulfide production of SRB grown for 24 hours with varying concentrations of MgO as represented as parts per billion (ppb) is shown in FIG. 2B. 90% confidence intervals were constructed using a parametric Bootstrap procedure based on a zero-inflated log-normal model where the parameters were estimated using maximum likelihood. The relationship between MgO dose level and the response (SRB Growth and H2S Concentration) was assessed with an F test based on a simple linear model. Data is presented as Mean±SEM.

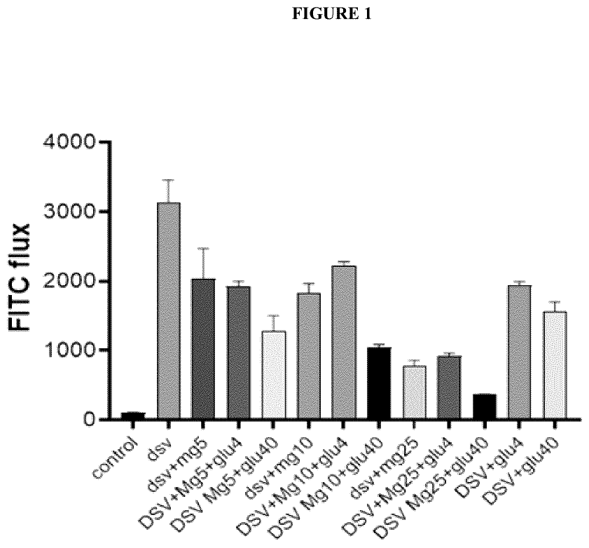

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” also includes two or more different compound. As used herein, the term “include” and its grammatical variants are intended to be non-limiting, such that recitation of items in a list is not to the exclusion of other like items that can be substituted or other items that can be added to the listed items.

The term “compound” or “agent”, as used herein, unless otherwise indicated, refers to any specific chemical compound or composition (such as a magnesium compound or a glutamine compound) as disclosed herein, and includes where relevant tautomers, regioisomers, geometric isomers as applicable, and also where applicable, stereoisomers, including diastereomers, optical isomers (e.g. enantiomers) thereof, as well as pharmaceutically acceptable salts and chelates thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers, where applicable, in context. The term also refers, in context to forms of compounds which have been modified to facilitate the administration and delivery of compounds to a site of activity.

The term “patient” or “subject” is used throughout the specification within context to describe an animal, generally a mammal, including a domesticated mammal including a farm animal (dog, cat, horse, cow, pig, sheep, goat, etc.) and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human.

The terms “effective” or “pharmaceutically effective” are used herein, unless otherwise indicated, to describe an amount of a compound or composition which, in context, is used to produce or affect an intended result, usually the inhibition or treatment of a disease state and/or condition such as metabolic syndrome, a disease state or condition associated with same as described herein and/or gut dysbiosis and/or leaky gut syndrome. In embodiments, the term synergistic effective amount refers to amounts of a magnesium compound and glutamine or salt thereof which produce a synergistic effect (i.e., an effect which is greater than additive) on metabolic syndrome, a disease state or condition related thereto as described herein and/or to dysbiosis and/or leaky gut syndrome.

The terms “treat”, “treating”, and “treatment”, etc., as used herein, refer to any action providing a benefit to a patient at risk for or afflicted by metabolic syndrome, or a related disease state and/or condition or gut dysbiosis and/or leaky gut syndrome as otherwise described herein. The benefit may be in curing the disease state or condition, inhibiting its progression, or ameliorating, lessening or suppressing one or more symptoms of metabolic syndrome or a related disease state or condition and gut dysbiosis and/or leaky gut symptoms caused by the disease state and/or condition. Treatment, as used herein, encompasses therapeutic treatment and in certain instances, prophylactic treatment, depending on context.

The term “co-administration”, “co-administered” or “combination therapy” is used to describe a therapy in which at least two active compounds in effective amounts are used to treat a disease state or condition as otherwise described herein, especially metabolic syndrome or insulin resistance, or a related disease state or condition in combination with dysbiosis and/or leaky gut syndrome either at the same time or within dosing or administration schedules defined further herein or ascertainable by those of ordinary skill in the art. Although the term co-administration preferably includes the administration of two active compounds to the patient at the same time (simultaneous or concurrent administration), although it is not necessary that the compounds be administered to the patient at the same time, although effective amounts of the individual compounds will be present in the patient at the same time. In addition, in certain embodiments, co-administration will refer to the fact that two compounds are administered at significantly different times, but the effects of the two compounds are present at the same time.

The present invention is directed to compositions and methods for treating metabolic syndrome, related disease states and/or conditions and gut dysbiosis (with or without leaky gut). Generally, the compositions include a compound that reduces H2S in the gut and protects the intestinal barrier (magnesium, glutamine). Metabolic syndrome is a well-known phenomenon responsible for multiple complications of the cardiac, vascular, hepatic, renal systems. This disclosure describes compounds that reduce sulfate-reducing bacteria (SRB) and/or H2S in the gut and decrease intestinal permeability of the intestinal tract damaged via SRB or gut-derived H2S as a prophylactic prior to disease progression and/or therapeutic treatment upon or after development of disease to reduce and treat associated complications.

A compound or combination of compounds (magnesium, glutamine) may be used to target SRBs and/or the gut-bacteria derived H2S and improve/reduce intestinal permeability that can lead to treatment of metabolic syndrome, insulin resistance and its complications such as cardiovascular disease and coronary heart disease, non-alcoholic fatty liver disease, non-alcohol steatohepatitis (NASH), fibrosis, including cirrhosis, high blood pressure, general and/or abdominal obesity, impaired fasting glucose, impaired insulin resistance/diabetes type 2, high triglyceride and LDL cholesterol (lipodystrophy), low HDL cholesterol, renal disease and failure, chronic obstructive pulmonary disease, among others.

Treatment can be administered prophylactically or therapeutically to limit severity and complications of metabolic syndrome and elated disease states and/or conditions.

Leaky gut is a well-established complication associated with obesity and insulin resistance as seen in metabolic syndrome. Increased presence of bacterial DNA is seen in patients with metabolic syndrome and type 2 diabetes leading to endotoxemia as seen by increased levels of plasma lipopolysaccharide.

The pharmaceutical composition according to the present invention may be formulated in different forms including oral, parenteral (e.g. transcutaneous, subcutaneous, intramuscular, intravenous, intraperitoneal, intradermal), buccal, rectal, topical (e.g. intranasal, intrapulmonary, intramammary, intravaginal, intrauterine). A pharmaceutical composition can be administered to a mucosal layer, such as, but not limited to, nasal and respiratory mucosa (via spray or aerosol), or oral mucosa with disintegrating or dissolving route. This can be administered via a sustained or delayed release. The composition can be formulated to be optimized for time of contact in the intestinal lumen with active agent targeting H2S-producing bacteria during periods of microbial fermentation after meals.

Agents may be provided in any suitable form including, but not limited to, tablet, capsule, emulsion, spray, aerosol, or mixture. Compounds may be delivered in formulation with carriers or vehicles for different modes of delivery. For example, this can be accomplished using topical dosage for cream, ointment, aerosol formulation, non-aerosol spray, gel, lotion, etc. Formulation may include additives such as skin penetrating enhancer, colorant, fragrance, flavoring, moisturizer, thickener, etc.

Formulations may be presented in unit dosage form and may be prepared by methods well known such as including a pharmaceutically acceptable carrier, additive or excipient that constitute additional ingredients. Formulations may be prepared by uniformly bringing the active compound into a liquid carrier, solid carrier, or both.

The amount of active agent administered can vary depending on factors such as weight, physical condition, age, route of administration. Thus, the absolute weight of the active agent included in a given unit dosage form can vary widely and depends on certain factors such as species, age, weight and condition of subject, route of administration. Accordingly, it is not practical to set forth generally the amount that constitutes an amount of the active agent effective for all possible applications. Those of ordinary skill in the art, however, can readily determine the appropriate amount with due consideration of such factors.

For instance, certain active agents may be administered at the same dose and frequency for which the active agent has received regulatory approval. In other cases, active agents may be administered at the same dose and frequency at which active agent is being evaluated in clinical and preclinical studies. One can alter the dosages and/or frequency as needed to achieve a desired level effect from the active agent. One can use standard/known regimens and/or customize dosing as needed.

The active agents principally act in the gut lumen, where SRB and/or bacteria-derived hydrogen sulfide is available; thus, gut luminal concentrations are often more relevant than blood concentrations. Thus, an effective amount of active agent may be significantly less than reported effective doses of the active agent for other indications since the active agents act directly in the gut rather than being absorbed and diluted in plasma. Multiple metal compounds used in organic and inorganic forms (magnesium) and glutamine amino acid are noted based on effect of bioavailability on blood concentrations. Since the active agents act in the gut lumen, prior to any physiologic absorption or metabolism, the effective concentration may be lower than listed, and lower than doses required for similar blood concentration bioavailability as reported in the literature.

The daily dose of active agents is provided as the mass of active agent (mg/day). For other active agents, daily dose is expressed as mass of active agent relative to mass of subject (mg/kg/day). The general dosage amounts that follow are provided as daily mass. Unless otherwise specified, the values provided for the mass of active agent in a minimum daily dose, maximum daily dose, or the endpoints for a range of daily dose values.

In some embodiments, the method can include administering sufficient active agent to provide a minimum daily dose (mg/day) of at least 0.001 mg such as, for example, at least 0.0012 mg, at least 0.0024 mg, at least 0.5 mg, at least 1 mg, at least 1.5 mg, at least 2 mg, at least 3 mg, at least 4 mg, at least 5 mg, at least 6 mg, at least 7 mg, at least 8 mg, at least 9 mg, at least 10 mg, at least 15 mg, at least 20 mg, at least 30 mg, at least 33 mg, at least 35 mg, at least 40 mg, at least 45 mg, at least 50 mg, at least 60 mg, at least 70 mg, at least 80 mg, at least 90 mg, at least 100 mg, at least 200 mg, at least 250 mg, at least 300 mg, at least 400 mg, at least 500 mg, at least 600 mg, at least 700 mg, at least 750 mg, at least 800 mg, at least 900 mg, or at least 1000 mg/kg.

In some embodiments, the method can include administering sufficient active agent to provide a maximum daily dose (mg/day) of no more than 8000 mg such as, for example, no more than 5000 mg, no more than 3150 mg, no more than 2000 mg, no more than 1600 mg, no more than 1000 mg/kg, no more than 900 mg, no more than 800 mg, no more than 700 mg, no more than 600 mg, no more than 500 mg, no more than 400 mg, no more than 300 mg, no more than 200 mg, no more than 195 mg, no more than 150 mg, no more than 100 mg, no more than 90 mg, no more than 80 mg, no more than 75 mg, no more than 70 mg, no more than 65 mg, no more than 60 mg, no more than 55 mg, no more than 50 mg, no more than 45 mg, no more than 40 mg, no more than 35 mg, no more than 30 mg, no more than 25 mg, no more than 20 mg, no more than 15 mg, or no more than 10 mg, no more than 3.6 mg, no more than 2 mg, no more than 1.5 mg, no more than 0.01 mg, or no more than 0.004 mg.

In some embodiments, the method can include administering sufficient active agent to provide a daily dose (mg/day) that falls within a range having endpoints defined by any minimum dose listed above and any maximum dose listed above that is greater than the minimum dose. For example, the method can include administering sufficient active agent to provide a dose of from 50 mg to 5000 mg or more, from 250 mg to 2000 mg, from 500 mg to 1000 mg, from 250 mg to 8000 mg, from 500 mg to 3150 mg, from 750 mg to 1600 mg, from 0.5 mg to 300 mg, from 10 mg to 60 mg, from 20 mg to 40 mg, from 15 mg to 2000 mg, from 30 mg to 300 mg, from 45 mg to 150 mg, from 20 mg to 195 mg, from 33 mg to 100 mg, from 40 mg to 60 mg, from 0.001 mg to 2 mg, from 0.0012 mg to 0.01 mg, or from 0.0024 mg to 0.004 mg.

In certain embodiments, the method can include administering sufficient active agent to provide a daily dose (mg/day) equal in value to any minimum daily dose or any maximum daily does listed above. Thus, for example, the method can include administering sufficient active agent to provide a daily dose (mg/day) of 0.5 mg, 1.5 mg, 3.6 mg, 10 mg, 15 mg, 40 mg, 60 mg, 100 mg, 150 mg, 250 mg, 300 mg, 500 mg, 750 mg, 1600 mg, 2000 mg, etc.

In exemplary embodiments in which the active agent magnesium sulfate, the method can include administering sufficient active agent to provide a dose of up to 20 g/day to 40 g/day.

In exemplary embodiments in which the active agent is magnesium carbonate, the method can include administering sufficient active agent to provide a dose of from 5 mL/day to 15 mL/day.

In exemplary embodiments in which the active agent is magnesium citrate, the method can include administering sufficient active agent to provide a dose of from 11.3 g/day to 17.45 g/day.

In exemplary embodiments in which the active agent is magnesium glucoheptonate, the method can include administering sufficient active agent to provide a dose of from 1500 mg/day to 9000 mg/day.

In exemplary embodiments in which the active agent is magnesium gluconate, the method can include administering sufficient active agent to provide a dose of 500 mg/day.

In exemplary embodiments in which the active agent is magnesium hydroxide, the method can include administering sufficient active agent to provide a dose of from 1200 mg/day to 4800 mg/day.

In exemplary embodiments in which the active agent is magnesium oxide, the method can include administering sufficient active agent to provide a dose of from 400 mg/day to 1600 mg/day.

In exemplary embodiments in which the active agent is magnesium L-aspartate hydrochloride, the method can include administering sufficient active agent to provide a dose of from 1230 mg/day to 2460 mg/day.

In exemplary embodiments in which the active agent is magnesium L-lactate, the method can include administering sufficient active agent to provide a dose of from 700 mg/day to 2800 mg/day.

In exemplary embodiments in which the active agent glutamine, including L-glutamine, the method can include administering sufficient active agent to provide a dose of from 5 g/day to 30 g/day, such as, for example, 0.5 g/kg/day.

A single dose may be administered all at once, continuously for a prescribed period of time, or in multiple discrete administrations. When multiple administrations are used, the amount of each administration may be the same or different. For example, a dose of 1 mg/day may be administered as a single administration of 1 mg, continuously over 24 hour, as two 0.5 mg administrations, or as a first administration of 0.75 mg followed by a second administration of 0.25 mg. When multiple administrations are used to deliver a single dose, the interval between administrations may be the same or different.

In some embodiments, the active agent may be administered, for example, from a single dose to multiple doses per week, although in some embodiments the method can involve a course of treatment that includes administering doses of the active agent at a frequency outside this range. When a course of treatment involves administering multiple within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In embodiments, the active agent often may be administered at least one per day such as, for example, once per day (QD), twice per day (BID), three times per day (TID), or four times per day (QID). In embodiments, the active may be administered more than four times per day. In certain embodiments, the active agent may be administered twice per day (BID). In some embodiments, the method can include administering active agent at a dosage up to three hours before a meal, with a meal, or up to three hours after a meal.

In some embodiments, the method can include administering active agent at a dosage and frequency listed above for a minimum period of at least three days such as, for example, at least five days, at least seven days, at least 10 days, at least 14 days, or at least 17 days. In some embodiments, the method can include administering active agent at a dosage and frequency listed above for a maximum period of no more than 30 days such as, for example, no more than 28 days, no more than 24 days, no more than 21 days, no more than 17 days, no more than 14 days, or no more than 10 days. In some embodiments, the method can include administering active agent at a dosage and frequency listed above for a period that falls within a range having endpoints defined by any minimum period listed above and any maximum period listed above that is great than the minimum period. Thus, in certain embodiments, the duration of treatment can be from three to 21 days. In some of these embodiments, the duration of treatment can be 10-17 days such as, for example, 14 days. In some embodiments, the method can involve continuing treatment for as long as symptoms and/or clinical signs persist can be repeated if symptom or clinical signs recur.

Treating a condition can be prophylactic or, alternatively, can be initiated after the subject exhibits one or more symptoms or clinical signs of the condition. Treatment that is prophylactic e.g., initiated before a subject manifests a symptom or clinical sign of the condition such as, for example, while an infection remains subclinical—is referred to herein as treatment of a subject that is “at risk” of having the condition. As used herein, the term “at risk” refers to a subject that may or may not actually possess the described risk. Thus, for example, a subject “at risk” of a condition is a subject possessing one or more risk factors associated with the condition such as, for example, genetic predisposition, ancestry, age, sex, geographical location, lifestyle, or medical history.

Accordingly, a composition can be administered before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

### EXAMPLES

Leaky gut is a common finding in nearly every chronic disease Leaky gut is considered the mechanism for chronic inflammation secondary to abnormal translocation of gut bacteria across the intestinal barrier. The cause of this abnormal increase in intestinal permeability leading to the leak of gut microbes was not known. Recently, we found that Desulfovibrio vulgaris (DSV), a common member of the hydrogen sulfide-producing sulfate reducing bacteria (SRB) family of rare resident gut bacteria causes leaky gut. SRB is a commensal bacteria that could act pathologically as a pathobiont when their numbers increase dramatically (SRB bloom) in the setting of a disruption of the gut microbiome (gut dysbiosis). Since SRB bloom is a characteristic feature of every type of gut dysbiosis we have tested (antibiotics, stress, legume lectins, high fat diet) and is seen in clinical conditions associated with gut dysbiosis, a bloom of SRB in the setting of gut dysbiosis may be the explanation for the leaky gut seen in all these settings.

To address this problem, the inventors tested and found that magnesium (Mg) was effective in protecting the intestinal barrier by suppressing SRB and inhibiting its metabolism (blocking production of hydrogen sulfide). A poorly absorbable form of magnesium (e.g. magnesium oxide, magnesium sulfate, magnesium chloride, magnesium gluconate and magnesium carbonate, mixtures thereof, among others) could then work well against SRB in the gut even without systemic entry as the desired effect is localized to the intestinal lumen. In contrast, an easily absorbable form of magnesium (e.g., magnesium-L-threonate, magnesium malate, magnesium citrate, magnesium diglycinate, magnesium acetyl taurate) would be more effective in achieving a systemic effect such as blocking pain triggered by bacteria-derived hydrogen sulfide as hydrogen sulfide works as a gaseous neurotransmitter in triggering the firing of pain pathways.

Glutamine has been reported to protect against the development of leaky gut in critical illness where a functional state of insufficient glutamine availability is common (Angela L. Morrison, Martin Dinges, Kristen D. Singleton, Kelli Odoms, Hector R. Wong, and Paul E. Wischmeyer. Glutamine's protection against cellular injury is dependent on heat shock factor-1 (Am J Physiol Cell Physiol290: C1625-C1632, 2006. First published Jan. 25, 2005; doi:10.1152/ajpcell.00635.2005.0363-6143/06; Paul E Wischmeyer Glutamine: role in gut protection in critical illness. Curr Opin Clin Nutr Metab Care 2006 September; 9(5):607-12. doi: 10.1097/01.mco.0000241672.09676.03).

It is not known, however, whether the combination of magnesium and glutamine is more effective in protecting against leaky gut than either compound alone. In this study, we tested the hypothesis that the novel combination of magnesium and glutamine may be more effective than magnesium alone or glutamine alone in protecting the intestinal barrier.

### Methods

In this first in-vitro study, the inventors compared the flux of a fluorescent probe (FITC-dextran) across the intestinal barrier in a short-term culture model system with Caco2 cells. FITC flux was measured by delivering the probe to the apical side of the epithelial layer and then measuring the appearance of the probe on the basolateral side of the cell culture. Normal intestinal barrier function is expected to resist the movement of a molecule the size of dextran through the epithelial layer. We tested FITC flux with different combinations of MgSo4 at 5, 10, 25 mM and glutamine at 4 and 40 mM. Caco2 cells were treated with Mg and/or glutamine for 24 hrs followed by DSV infection for 20 hrs.
1. Caco2 cells were grown for 21 days in 12-well transwell plates (in DMEM+ 20% FBS). This would allow for differentiation and polarization of Caco2 cells to become small intestinal enterocyte-like cells with apical and basolateral surface. Medium was changed every 3-4 days and prior to the experiment. Transepithelial electric resistance (TEER) was measured with EVOM meter (WPI, Sarasota, FL). Cells with TEER >450 W (high resistance means good intestinal barrier function) were used in the experiment.
2. MgSO4 was prepared in sterile water and added at a final concentration of 5 mM, 10 mM or 25 mM. Glutamine was added at a final concentration of 4 mM or 40 mM. Both compounds were either added alone or together to both apical and basolateral surface of the transwell for 24 hrs.
3. Next day, to the pretreated cells, Desulfovibrio vulgaris Hildenborough (DSV, ATCC 29579) was added to the apical surface at the MOI of 40 for 18 hrs.
4. On the following day, 25 mg/ml of Fluorescein isothiocyanate-dextran 4000 (FITC-DextranxFD-4, Sigma-Aldrich) was made in sterile PBS. 40 ml of this solution was added apically to the cells post infection. After 1 hour of incubation at 370 C, 100 ml of the basolateral medium was collected and analyzed for the presence of FITC fluorescence. Fluorescence was measured at the wavelengths of excitation and emission at 485 and 520 nm, respectively.

### Method 2

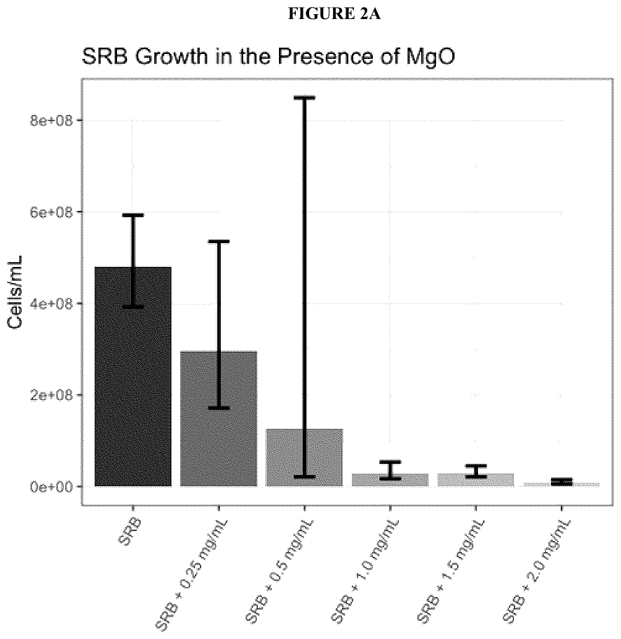

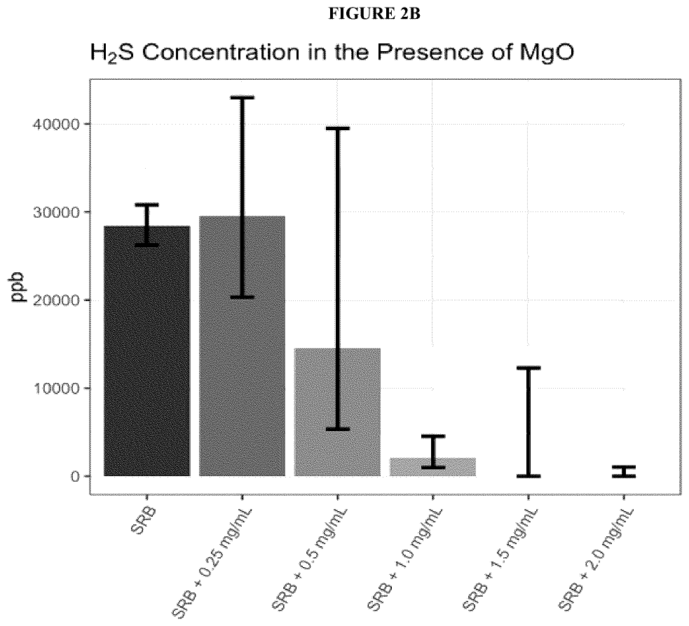

In this second in-vitro experiment, bacteria was grown with varying doses of MgO (0.5-2.0 mg/ml) in liquid media at 37° for 24 hours (n=3). Total counts were obtained and represented as cells per ml (FIG. 2A). To account for the heavy right skew of the response variable, the analysis was conducted using a shifted log-transformation of the data and 90% confidence intervals were constructed using Cox's method. Hydrogen sulfide production of SRB grown for 24 hours with varying concentrations of MgO as represented as parts per billion (ppb) (FIG. 2B). 90% confidence intervals were constructed using a parametric Bootstrap procedure based on a zero-inflated log-normal model where the parameters were estimated using maximum likelihood. The relationship between MgO dose level and the response (SRB Growth and H2S Concentration) was assessed with an F test based on a simple linear model Data is presented as Mean±SEM.

Exemplary Dosing Schedule of Magnesium Compound(s)
With Glutamine Based Upon Example
Converting mM to g
g = moles × atomic mass = moles × (g/mol)
MgSO4 = 120.366 g/mol
MgSO4

| milliMole | Mole | g = moles × (g/mol) | Grams |
|---|---|---|---|
| 5 mM | =0.005M | g = 0.005 × (120.366 g/mol) | =0.6 |
| 10 mM | =0.01M | g = 0.01 × (120.366 g/mol) | =0.12 |
| 25 mM | =0.025M | g = 0.025 × (120.366 g/mol) | =3.01 |

Glutamine = 146.14 g/mol
Glutamine

| milliMole | Mole | g = moles × (g/mol) | grams |
|---|---|---|---|
| 4 mM | =0.004M | g = 0.004 × (146.14 g/mol) | =0.586 |
| 40 mM | =0.040M | g = 0.040 × (146.14 g/mol) | =5.726 |

Combination of MgSO2 at ratio consistent
with 25 mM + glutamine at 40 mM per day

| | | | at ⅓ | Rounded up |
|---|---|---|---|---|
| | | | dose | for ease of |
| | gram | milligram | (mg) | measurement |
| MgSO4 | 3 | 3000 | 1000 | 1000 |
| glutamine | 5.7 | 5700 | 1900 | 2000 |

| | | |
|---|---|---|
| a.m. dose | p.m. dose | Total daily |
| (mg) | (mg) | dose (mg) |

Suggested dosing

| | | | |
|---|---|---|---|
| MgSO4 | 500 | 500 | 1000 |
| glutamine | 1000 | 1000 | 2000 |

Possible other dose permutations for the combination to be given b.i.d.

| | | | |
|---|---|---|---|
| MgSO4 | 100 | 100 | 200 |
| glutamine | 200 | 200 | 400 |
| MgSO4 | 200 | 200 | 400 |
| glutamine | 400 | 400 | 800 |
| MgSO4 | 250 | 250 | 500 |
| glutamine | 500 | 500 | 1000 |
| MgSO4 | 400 | 400 | 800 |
| glutamine | 800 | 800 | 1600 |

Possible other dose permutations for the
combination to be given t.i.d. with meals

| | a.m. dose | Mid-day dose | p.m. dose | Total daily |
|---|---|---|---|---|
| | (mg) | (mg) | (mg) | dose (mg) |
| MgSO4 | 400 | 100 | 400 | 900 |
| glutamine | 800 | 200 | 800 | 1800 |
| MgSO4 | 400 | 200 | 400 | 1000 |
| glutamine | 800 | 400 | 800 | 2000 |
| MgSO4 | 500 | 200 | 500 | 1200 |
| glutamine | 1000 | 400 | 1000 | 2400 |
| MgSO4 | 500 | 250 | 500 | 1250 |
| glutamine | 1000 | 500 | 1000 | 2500 |
| MgSO4 | 500 | 500 | 500 | 1550 |
| glutamine | 1000 | 1000 | 1000 | 3000 |

Any combination of the doses above using different magnesium formulation with variable absorbability along with glutamine are described as additional embodiments in the present application.

1. A composition formulated in one or two parts comprising at least one magnesium compound in combination with glutamine or a pharmaceutically acceptable salt thereof wherein said magnesium compound and said glutamine or pharmaceutically acceptable salt thereof comprises in combination an effective amount for treating metabolic syndrome or a related disease state, condition or symptom thereof.

2. The composition according to claim 1 comprising an amount of said magnesium compound ranging from 5 mM to 50 mM (about 0.6 to 6.0 grams) and an amount of glutamine ranging from 4 mM to 50 mM (about 0.586 to 7.5 grams).

3. The composition according to claim 1 wherein said magnesium compound comprises a low bioavailability magnesium compound and a high bioavailability magnesium compound.

4. The composition according to claim 3 wherein said low bioavailability magnesium compound is magnesium oxide, magnesium hydroxide, magnesium glucoheptonate, magnesium gluconate, magnesium chloride, magnesium sulfate, magnesium carbonate or a mixture thereof and said high bioavailability magnesium compound is magnesium-L-threonate, magnesium malate, magnesium lactate, magnesium aspartate, magnesium citrate, magnesium diglycinate, magnesium acetyl taurate or a mixture thereof.

5. The composition according to claim 1 wherein said magnesium compound is magnesium sulfate in combination with glutamine or a pharmaceutically acceptable salt thereof.

6. The composition according to claim 1 wherein said magnesium compound(s) and said glutamine or glutamine salt are included in same composition in a molar ratio ranging from about 0.35 to about 1.0 magnesium to each 1.0 mole of glutamine.

7. The composition according to claim 1 wherein said magnesium compound(s) and said glutamine or glutamine salt are included in same composition in a molar ratio ranging from about 0.6 to about 0.7 moles of magnesium to each 1.0 mole of glutamine.

8. The composition according to claim 1 wherein said magnesium compound(s) and said glutamine or glutamine salt are included in same composition in a molar ratio ranging from about 0.625 moles of magnesium to each 1.0 mole of glutamine.

9. The composition according to claim 1 wherein said composition is formulated into a single composition for oral administration.

10. A method of treating metabolic syndrome or a related disease state, condition or symptom thereof and gut dysbiosis, comprising administering to a patient or subject in need an effective amount of at least one magnesium compound in combination with an effective amount of glutamine or a pharmaceutically acceptable salt thereof.

11. The method according to claim 10 wherein said magnesium compound is a mixture of a low bioavailability magnesium compound and a high bioavailability magnesium compound.

12. The method according to claim 11 wherein said low bioavailability magnesium compound is magnesium oxide, magnesium hydroxide, magnesium glucoheptonate, magnesium gluconate, magnesium chloride, magnesium sulfate, magnesium carbonate or a mixture thereof and said high bioavailability magnesium compound is magnesium-L-threonate, magnesium malate, magnesium lactate, magnesium aspartate, magnesium citrate, magnesium diglycinate, magnesium acetyl taurate or a mixture thereof.

13. The method according to claim 10 wherein said magnesium compound is magnesium sulfate.

14. The method according to claim 10 wherein said magnesium compound(s) is administered to said patient or subject in an amount ranging from 5 mM to 50 mM (about 0.6 to 6.0 grams) and said glutamine or a pharmaceutically acceptable salt thereof is administered to said patient or subject in an amount ranging from 4 mM to 50 mM (about 0.586 to 7.5 grams).

15. A method of treating a disease state and/or condition selected from the group consisting of metabolic syndrome, insulin resistance, impaired glucose metabolism, insulin resistance, dysregulation of blood pressure and dyslipidemia, cardiovascular disease, diabetes (especially diabetes II), cerebrovascular disease, fatty liver disease, non-alcoholic steatohepatitis (NASH) and fibrosis in combination with gut dysbiosis, comprising administering to a patient or subject in need an effective amount of at least one magnesium compound in combination with an effective amount of glutamine or a pharmaceutically acceptable salt thereof.

16. The method according to claim 15 wherein said magnesium compound is a mixture of a low bioavailability magnesium compound and a high bioavailability magnesium compound.

17. The method according to claim 16 wherein said low bioavailability magnesium compound is magnesium oxide, magnesium hydroxide, magnesium glucoheptonate, magnesium gluconate, magnesium chloride, magnesium sulfate, magnesium carbonate or a mixture thereof and said high bioavailability magnesium compound is magnesium-L-threonate, magnesium malate, magnesium lactate, magnesium aspartate, magnesium citrate, magnesium diglycinate, magnesium acetyl taurate or a mixture thereof.

18. The method according to claim 15 wherein said magnesium compound is magnesium sulfate.

19. The method according to claim 15 wherein said magnesium compound(s) is administered to said patient or subject in an amount ranging from 5 mM to 50 mM (about 0.6 to 6.0 grams) and said glutamine or a pharmaceutically acceptable salt thereof is administered to said patient or subject in an amount ranging from 4 mM to 50 mM (about 0.586 to 7.5 grams).

20. The method according to claim 15 wherein said disease state and/or condition is metabolic syndrome or insulin resistance.
